(19) United States
(12) Patent Application Publication
(10) Pub. No.: US 2015/0072381 A1
(43) Pub. Date: Mar. 12, 2015

(54) HOMOLOGOUS RECOMBINATION-BASED NUCLEIC ACID MOLECULAR CLONING METHOD AND RELATED KIT

(71) Applicant: SHENZHEN ZHONGLIAN BIOTECHNOLOGY CO., LTD, Shenzheng (Guangdong) (CN)

(72) Inventor: Haoyang Yu, Shenzhen City (CN)

(21) Appl. No.: 14/499,257

(22) Filed: Sep. 29, 2014

Related U.S. Application Data
(63) Continuation-in-part of application No. PCT/CN2013/073186, filed on Mar. 26, 2013.

(30) Foreign Application Priority Data
Mar. 30, 2012 (CN) 201210090049.1

(51) Int. Cl. C12N 15/66

(52) U.S. Cl. CPC C12N 15/66
USPC 435/91.41; 435/188

(57) ABSTRACT

This invention provides a homologous recombination-based nucleic acid molecular cloning method. According to the method of the present invention, a target DNA is cloned into a vector through homologous recombination by providing a linearized vector with both ends respectively added with a sequence (namely, a target DNA-specific homologous arm) homologous with sequences of both ends of the target DNA or a flank sequence thereof, or by utilizing a ligation fragment containing both the target DNA-specific homologous arm and a vector-specific homologous arm (a sequence homologous with a specific region of the vector). The method of the present invention is especially applicable to the cloning of a large DNA fragment and to the studies of single nucleotide polymorphism. The present invention further provides a related kit.

### CROSS-REFERENCE TO RELATED APPLICATIONS

This application is a continuation-in-part of International Patent Application No. PCT/CN2013/073186 with an international filing date of Mar. 26, 2013, designating the United States, now pending, and further claims priority benefits to Chinese Patent Application No. 201210090049.1 filed Mar. 30, 2012. The contents of all of the aforementioned applications, including any intervening amendments thereto, are incorporated herein by reference.

### CORRESPONDENCE ADDRESS

Inquiries from the public to applicants or assignees concerning this document should be directed to: WAYNE & KING LLC, P.O. BOX 439, PAINTED POST, NY 14870.

### SEQUENCE LISTING

Applicant attaches the paper copy of the Sequence Listing in a separate list.

### BACKGROUND OF THE INVENTION

1. Technical Field

The present application relates to the technical field of DNA recombination. More specifically, the present application relates to a homologous recombination-based nucleic acid molecular cloning method, applications thereof and a related kit.

2. Background Art

In the study of molecular biology and in biotechnology industry, there is always a need to clone a desired DNA molecule into a vector, particularly at a specific location of the vector.

A conventional method for cloning a target DNA into a vector, such as a plasmid, at a predetermined location usually includes six major steps: (1) cleaving the vector DNA with a restriction endonuclease, and purifying the linearized vector; (2) treating the linearized vector with Calf Intestinal Phosphatase (CIP) to minimize the level of self-recircularization of the linearized vector during ligation; (3) amplifying the target DNA by a polymerase chain reaction (PCR) using PCR primers, wherein the primers will add to the 5′- and 3′-ends of the amplified target DNA enzyme recognition sites of the restriction endonuclease used to linearize the vector DNA; (4) cleaving the amplified target DNA with the restriction endonuclease used to linearize the vector DNA, and then purifying the cleaved target DNA; (5) ligating the purified target DNA and the purified linearized vector using a DNA ligase; and (6) transforming a ligation product into a host cell, such as a competent Escherichia coli cell, and then selecting a transformed cell containing the desired cloning product, wherein the target DNA is inserted into the vector at a desired cloning site. The conventional cloning method is tedious and time-consuming. It has relatively low cloning efficiency, and is also limited by the availability of suitable restriction enzyme recognition sites on the vector and the target DNA.

Homologous recombination may be utilized to significantly improve the efficiency of gene cloning. At present, there have been various homologous recombination-based cloning methods. The common practice is as follows: a target DNA is amplified by PCR, and sequences homologous with both ends of a linearized vector DNA are added to both ends of the amplified target DNA utilizing the PCR primers; then cloning the PCR primers into a vector by homologous recombination, or co-transforming or co-transfecting the linearized vector and the PCR product into a host cell by the action of enzymes in vitro, and cloning the PCR primers into the vector by homologous recombination under the action of enzymes in vivo.

However, there are still some problems in the existing homologous recombination-based cloning methods, particularly in the condition of cloning the large fragment genomic DNA from eukaryotes or studying single nucleotide polymorphism (SNP) in human genomic DNA. For example, it is still difficult to amplify more than 10 kb of the large fragment genomic DNA by PCR currently. As for in vivo homologous recombination, the involvement of co-transforming or co-transfecting the target DNA fragments amplified by the PCR and the vector DNA molecules into host cells results in low conversion rates. In addition, during the SNP study, it is difficult to distinguish whether the detected single nucleotide mutation is present in the genome itself or introduced artificially by PCR amplification. Therefore, new cloning methods capable of solving these problems are still needed.

The present application solves the abovementioned problems by providing a homologous recombination-based nucleic acid molecular cloning method.

### SUMMARY OF THE INVENTION

One aspect of the present application provides a homologous recombination-based nucleic acid molecular cloning method. In an embodiment, the present application provides a method of cloning a target DNA into a vector, the method comprising:

(a) adding to both ends of a linearized vector a first sequence and a second sequence, respectively, wherein the first sequence has a sequence that is homologous with a sequence of a first end of the target DNA or a flank sequence thereof, the second sequence has a sequence that is homologous with a sequence of a second end of the target DNA or a flank sequence thereof, an extended linearized vector with both ends respectively having the first sequence and the second sequence is obtained, and each of the first sequence and the second sequence, independently, has a length of at least 12 nucleotides, preferably 15 to 50 nucleotides, more preferably 35 to 50 nucleotides; and

(b) bringing the extended linearized vector into contact with a sample containing the target DNA, and cloning the target DNA into the vector by homologous recombination.

In a preferred embodiment, step (a) may be carried out as follows:

(i) providing a first primer, a second primer and a vector, wherein the first primer comprises the first sequence as the 5′-end and a sequence as the 3′-end that is specific to a first region of the vector, the second primer comprises the second sequence as the 5′-end and a sequence as the 3′-end that is specific to a second region of the vector, the sequence that is specific to the first region of the vector is preferably a sequence complementary to the first region of the vector, and the sequence that is specific to the second region of the vector is preferably a sequence complementary to the second region of the vector; and

(ii) bringing the first primer and the second primer into contact with the vector as a template, and obtaining the extended linearized vector with both ends respectively having the first sequence and the second sequence by a polymerase chain reaction.

The vector as the template in the polymerase chain reaction may be a linearized vector, and the first and second regions are preferably a first end and a second end of the linearized vector, respectively. The vector as the template in the polymerase chain reaction may also be a circular vector, and the polymerase chain reaction is preferably carried out in the presence of a helicase.

In another preferred embodiment, the step (a) may be carried out as follows:

(i) providing a first ligation fragment, a second ligation fragment and a vector, wherein the first ligation fragment has a sequence homologous with the sequence of the first end of the target DNA or the flank sequence thereof and a sequence homologous with the first region of the vector, and the second ligation fragment has a sequence homologous with the sequence of the second end of the target DNA or the flank sequence thereof and a sequence homologous with the second region of the vector; and

(ii) bringing the first ligation fragment and the second ligation fragment into contact with the vector, and obtaining the extended linearized vector with both ends respectively having the first sequence and the second sequence by homologous recombination.

In another embodiment, the present application provides a method of cloning a target DNA into a vector, the method comprising:

(a) providing a first ligation fragment, a second ligation fragment and a vector, wherein the first ligation fragment has a sequence homologous with a first region of the vector and a sequence homologous with a sequence of a first end of the target DNA or a flank sequence thereof, and the second ligation fragment has a sequence homologous with a second region of the vector and a sequence homologous with a flank sequence of a second end of the target DNA; and

(b) bringing a first DNA fragment and a second DNA fragment into contact with a linearized vector and a sample containing the target DNA, and cloning the target DNA into the vector by homologous recombination.

In the abovementioned embodiments, the homologous recombination may be carried out in the presence of an exonuclease and a single-stranded DNA binding protein or an annealing protein or an enzyme that has functions equivalent thereto. The exonuclease is preferably selected from Escherichia coli exonuclease I, Escherichia coli exonuclease III, Escherichia coli exonuclease VII, lambda bacteriophage exonuclease, T7 bacteriophage exonuclease, Redα, RecE and a mixture thereof. The single-stranded DNA binding protein or the annealing protein is preferably selected from extreme thermostable single-stranded DNA binding protein (ET SSB), RecA, T4 Gene 32 Protein, Thermus thermophilus RecA (Tth RecA), Escherichia coli single-stranded DNA binding protein (SSB), Redβ, RecT and a mixture thereof.

The homologous recombination may be carried out in any combination of the exonuclease and the single-stranded DNA binding protein or the annealing protein. In a preferred embodiment, the homologous recombination is carried out in the presence of RecE and RecT. In another preferred embodiment, the homologous recombination is carried out in the presence of Redα and Redβ. In a further preferred embodiment, the homologous recombination is carried out in the presence of RecE, RecT, Redα and Redβ.

The homologous recombination may further be carried out in the presence of other enzymes. The other enzymes are, for example, helicases, nucleic acid repair proteins, and the like.

In a further embodiment, the present application provides a kit for cloning a target DNA into a vector, the kit comprising:

(a) an enzyme mixture comprising an exonuclease and a single-stranded DNA binding protein or an annealing protein; and

(b) a reaction buffer.

The exonuclease may be a prokaryote exonuclease or a virus exonuclease, and preferably selected from Escherichia coli exonuclease I, Escherichia coli exonuclease III, Escherichia coli exonuclease VII, lambda bacteriophage exonuclease, T7 bacteriophage exonuclease, Redα, RecE and a mixture thereof.

The single-stranded DNA binding protein or the annealing protein may be selected from extreme thermostable single-stranded DNA binding protein (ET SSB), Rec A, T4 Gene 32 Protein, Thermus thermophilus RecA (Tth RecA), Escherichia coli single-stranded DNA binding protein (SSB), Redβ, RecT and a mixture thereof.

The enzyme mixture may comprise any combination of the exonuclease and the single-stranded DNA binding protein or the annealing protein. In a preferred embodiment, the enzyme mixture comprises RecE and RecT. In another preferred embodiment, the enzyme mixture comprises Redα and Redβ. In a further preferred embodiment, the enzyme mixture comprises RecE, RecT, Redα and Redβ.

The enzyme mixture may further contain a helicase and/or a nucleic acid repair protein. In a preferred embodiment, the enzyme mixture comprises an exonuclease, a helicase, a single-stranded DNA binding protein or an annealing protein, and a nucleic acid repair protein.

In a kit of the present application, the reaction buffer preferably comprises 1 to 10 mg/mL of Tris, 1 to 10 mg/mL of NaCl, 0.1 to 10 mg/mL of EDTA, 0.1 to 10 mg/mL of MgCl₂, 10 to 200 mg/mL of glycerol, 10 to 50 mg/mL of bovine serum albumin (BSA), 0.1 to 10 mg/mL of ATP, 1 to 10 mg/mL of Na₂HPO₄, 0.1 to 10 mg/mL of KH₂PO₄, and 0.1 to 10 mg/mL of dithiothreitol (DTT); the pH value is about 6.8 to about 7.4.

### BRIEF DESCRIPTION OF THE DRAWINGS

FIG. 1 is a schematic diagram of an embodiment of the method of the present application, wherein SM is a selection marker.

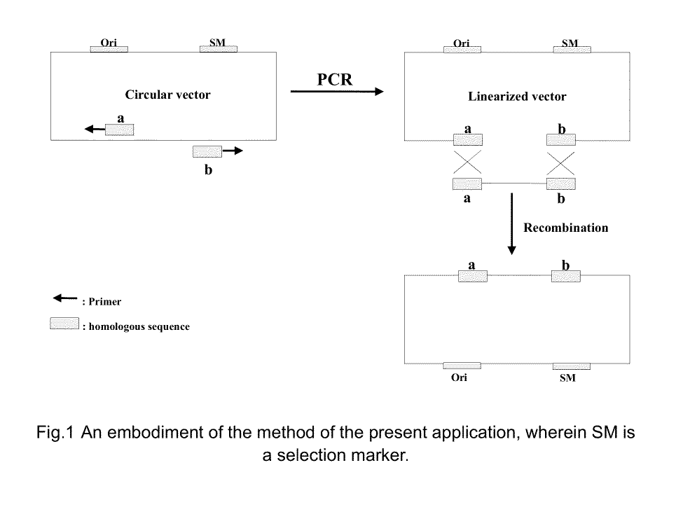

FIG. 2 is a schematic diagram of another embodiment of the method of the present application, wherein SM is a selection marker.

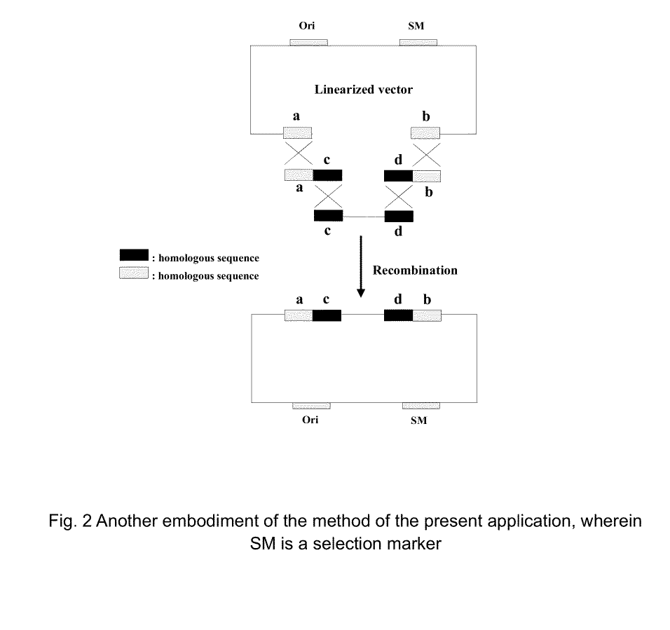

FIG. 3 is a schematic diagram showing positions of Fragments I, II, II and IV (Fragments 2-4 respectively) of a DHRS4 gene on the full-length DHRS4 gene (Fragment 1).

FIG. 4 is an electrophoretogram of a PCR product of a positive (ampicillin-resistant) cloned plasmid. Lane 1 is the amplified full-length DHRS4 gene (Fragment 1), Lanes 2-4 are Fragments 2-5, respectively, and Lane M is a DNA molecular weight marker.

FIG. 5 is a PstI restriction map. In FIG. A, Lane 1 is a DNA molecular weight marker, and Lane 2 is a standard restriction map of a murine TFIIA gene. In FIG. B, Lane 1 is a DNA molecular weight marker, and Lanes 2-14 are restriction maps of PCR amplification products with the plasmid DNA extracted from positive clones (transformants) as the template.

### DETAILED DESCRIPTION OF THE INVENTION

Unless defined otherwise, all scientific and technical terms used in the present application have the same meaning as commonly understood by persons skilled in the art to which the present application pertains.

It is found by the inventors that the abundance of a target DNA in a prepared DNA sample (e.g., an extracted genomic DNA sample) is sufficient to allow the target DNA to be cloned into a vector in vitro by homologous recombination without amplifying the target DNA prior to introduction of the target DNA into the vector, thus accomplishing the present application.

In the method of the present application, the target DNA is cloned into the vector by homologous recombination by providing a linearized vector with two ends respectively added with a sequence (namely, a target DNA-specific homologous arm) homologous with sequences of both ends of the target DNA or a flank sequence thereof, or by utilizing a ligation fragment containing both the target DNA-specific homologous arm and a vector-specific homologous arm (a sequence homologous with a specific region of the vector).

With the method of the present application, as there is no need to carry out PCR amplification on the target DNA, artificial mutation will not be introduced, the size limit of the target DNA (i.e., the fragment to be amplified) during the PCR amplification will not occur, and more than 10 kb (e.g., 10 to 100 kb) of the large DNA fragment can be cloned. Moreover, as the method of the present application is concerned with introduction of the target DNA into the vector in vitro, the problem of low conversion rates caused by co-transformation or co-transfection in in vivo recombination is thus solved. Accordingly, the present application solves the existing problems in the prior art by providing this method.

Vector

As used in the present application, the term “vector” refers to such an nucleic acid that is capable of transporting another ligated nucleic acid. The vector may be any vector, such as a plasmid, a cosmid, a virus or the like, or may also be a bacterial artificial chromosome (BAC) or a yeast artificial chromosome (YAC) and a phagemid. The vector may be an autonomously replicating vector or an integrating vector. The autonomously replicating vector is capable of autonomous replication in a host cell into which it is introduced, e.g., a bacterial vector having a bacterial replication origin and an mammalian episomal vector. The integrating vector is integrated into the genome of a host cell upon introduction into the host cell, and is thereby replicated along with the host genome, e.g., a mammalian non-episomal vector. Furthermore, some vectors, i.e., expression vectors, can direct the expression of genes to which they are effectively ligated. The vectors of the present application may further be vectors specially designed to allow DNA cloning in different hosts or allow DNA shuttling between hosts, i.e., shuttle vectors. The above-mentioned vectors are known to persons skilled in the art, and can be selected according to requirements, e.g., according to the host cells as used and according to the method of adding a first sequence and a second sequence as used in the present application.

The vectors of the present application may contain various elements for cloning, expression and screening. In an embodiment, the vectors contain replication origins for replication in host cells, such as ColE1 replication origins for replication in Escherichia coli host cells, 2μ replication origins for replication in yeast host cells, or virus replication origins, such as SV40 replication origins.

In another embodiment, the vectors contain selection markers for selecting correct recombinants, e.g., drug-resistance genes. The drugs are such as but not limited to ampicillin, streptomycin, kanamycin, chloramphenicol, hygromycin, methotrexate, etc. The selection markers may also be reporter genes such as but not limited to genes encoding a green fluorescent protein (GFP), β-galactosidase, luciferase, chloramphenicol acetyltransferase, β-glucuronidase, neomycin phosphotransferase and so on. According to the present application, marker genes that are differentially expressed in the original vectors and recombined vectors may also be used. The transformed host cells containing the recombined vectors may be readily identified using various methods known in the art, for example, may be identified by PCR amplifying (PCR screening) the target DNA fragments on the vectors contained in the positive clones.

The Target DNA can be cloned into a vector at any predetermined location. The cloning location can be selected according to the requirements. According to the selected cloning location and direction, the first region and the second region of the vector and sequences corresponding thereto in the primers and the ligation fragment of the present invention can be easily determined.

In an embodiment of the present application, the vector is a plasmid, and the predetermined location for insertion of the target DNA may be located at a restriction site, or between two restriction sites. A linearized vector can be obtained by cleaving the plasmid with one or more than one restriction endonuclease.

Target DNA

With the method of the present application, any target DNA may be cloned into the vector. The target DNA molecules may be derived from prokaryotes, such as bacterial genomic DNA and cDNA, or may be derived from eukaryotes, such as genomic DNA and cDNA of yeasts, mammals (e.g., humans) and the like, or may further be a DNA fragment mediated by a PCR reaction, a DNA fragment in a prokaryotic or eukaryotic genomic DNA library constructed by bacterial artificial chromosomes (BAC), yeast artificial chromosomes (YAC), cosmids and the like. The target DNA may be a gene encoding a protein, a sequence carrying gene mutations or lesions, or the like.

The target DNA may be a small fragment DNA, or may be a large fragment DNA. The method of the present application is particularly suitable for the cloning of a large fragment DNA, e.g., a large fragment DNA from 10 kb to 100 kb. The large fragment DNA may be a large fragment DNA from 10 kb to 100 kb, e.g., 10, 15, 20, 25, 30, 35, 40, 45, 50, 55, 60, 65, 70, 75, 80, 85, 90, 95 or 100 kb.

Preparation of the Extended Linearized Vector

According to the present application, an extended linearized vector is prepared by adding to both sides of the linearized vector a first sequence and a second sequence, respectively, wherein the first sequence comprises a sequence homologous with the sequence of the first end of the target DNA or the flank sequence thereof, and the second sequence comprises a sequence homologous with the sequence of the second end of the target DNA or a flank sequence thereof, so that the linearized vector with both sides respectively added with the first sequence and the second sequence can be ligated to the target DNA by homologous recombination. The first sequence and the second sequence can be the sequences that are homologous with the sequences of the corresponding ends of the target DNA or the flank sequences thereof.

The so-called “homologous” means that two nucleotide sequences have certain sequence identity or homology therebetween, so that they can be ligated by homologous recombination under the action of recombinases or recombination systems. The “homologous” includes, but is not limited to having at least 80% sequence identity between two nucleotide sequences, for example, having 85%, 90%, 91%, 92%, 93%, 94%, 95%, 96%, 97%, 98%, 99% or 100% identity. The term “sequence identity” is well-known to persons skilled in the art, meaning the percentage at which the nucleotides or amino acid residues of two nucleotide sequences or polypeptide sequences are identical during optimal alignment and analysis. The calculation method of the sequence identity is well-known to persons skilled in the art.

The homology of the first sequence and the second sequence as the homologous arms to the corresponding sequences of the target DNA is not limited as long as the extended linearized vector as obtained can carry out homologous recombination with the target DNA. Preferably, the first sequence has 100% sequence identity to the sequence of the first end of the target DNA or the flank sequence thereof and, preferably, the second sequence has 100% sequence identity to the sequence of the second end of the target DNA or the flank sequence thereof.

Each of the first sequence and the second sequence, independently, has a length of at least 12 nucleotides, preferably 15 to 50 nucleotides, e.g., 20, 25, 30, 35, 40, 45 or 50 nucleotides, more preferably 35 to 50 nucleotides.

The sequences and lengths of the first sequence and the second sequence can be designed based on the sequences of both ends of the target DNA or the flank sequences thereof according to the enzymes used in homologous recombination.

The first sequence and the second sequence can be added to both sides of the linearized vector through various methods. In a preferred embodiment, the first sequence and the second sequence can be respectively added to both sides of the linearized vector by PCR, using a first primer containing the first sequence and a vector-specific sequence and a second primer containing the second sequence and the vector-specific sequence, and using the vector as a template. For example, the first primer contains the first sequence and a sequence that is specific to the first region of the vector, and the second primer contains the second sequence and a sequence that is specific to the second region of the vector. For example, the PCR primers can be designed to have a 15-50 bp homologous sequence derived from the target DNA as the 5′-end and a subsequent 18-25 bp plasmid DNA template-specific primer sequence as the 3′-end. The plasmid DNA template-specific primer sequence in the PCR primers may be a sequence complementary to the plasmid DNA template. The so-called “complementary” means 100% complementary.

The PCR reaction can be carried out using any methods well-known in the art. The conditions of the PCR reaction can be selected or optimized by conventional experimentation. Please refer to, for example, Joseph Sambrook et al., Molecular Clonning: A Laboratory Manual, 3rd ed. Cold Spring Harbor Laboratory Press, 2001, and Carl W. Dieffenbach and Gabriela S. Dveksler, PCR primer: A Laboratory Manual, Cold Spring Harbor Laboratory Press, 1995.

The vector may be a linearized vector, or may be a circular vector.

In another embodiment, with the advantage of ligation fragments containing both the target DNA-specific homologous arm and the vector-specific homologous arm, such as the first ligation fragment and the second ligation fragment, instead of the PCR reaction, the ligation fragments are brought into contact with the vector to obtain an extended linearized vector by homologous recombination, i.e., a linearized vector with both sides added with the first sequence and the second sequence, respectively. In this case, the ligation fragments contain both the target-specific homologous arm and the vector-specific homologous arm. That is, the ligation fragments contain both the sequences homologous with the sequences of the ends of the target DNA or the flank sequences thereof, and the sequences homologous with the vector-specific regions. For example, the first ligation fragment contains a sequence homologous with the sequence of the first end of the target DNA or the flank sequence thereof and a sequence homologous with the first region of the vector, and the second ligation fragment contains a sequence homologous with the sequence of the second end of the target DNA or the flank sequence thereof and a sequence homologous with the second region of the vector. The vector can be a linearized vector or a circular vector. When the vector is a circular vector, the homologous recombination reaction can be carried out under the action of helicases.

The homologous recombination technology is well-known in the art. Homologous recombination can be implemented using any methods, any suitable enzymes, enzyme mixtures or enzyme systems known in the art. For example, the method hereinafter may be used so that the first sequence and the second sequence are added to both sides of the linearized vector by homologous recombination.

Enzyme

Homologous recombination may be implemented using any known enzymes, enzyme mixtures or enzyme systems for homologous recombination.

The method of the present application may be implemented using an enzyme mixture. The enzyme mixture may comprise an exonuclease and a single-stranded DNA binding protein or an annealing protein, or an enzyme or a protein that functions in substantially the same manner as the abovementioned enzymes. The exonuclease may be a prokaryote exonuclease or a virus exonuclease, or may be selected from Escherichia coli exonuclease I, Escherichia coli exonuclease III, Escherichia coli exonuclease VII, lambda bacteriophage exonuclease, T7 bacteriophage exonuclease, Redα, RecE and a mixture thereof. The single-stranded DNA binding protein or the annealing protein may be selected from extreme thermostable single-stranded DNA binding protein (ET SSB), RecA, T4 Gene 32 Protein, Thermus thermophilus RecA (Tth RecA), Escherichia coli single-stranded DNA binding protein (SSB), Redβ, RecT and a mixture thereof. The enzyme mixture may contain any combination of the exonucleases and the single-stranded DNA binding proteins or the annealing proteins mentioned above.

In a preferred embodiment, the enzyme mixture comprises RecE and RecT. In another preferred embodiment, the enzyme mixture comprises Redα and Redβ. In a further preferred embodiment, the enzyme mixture comprises RecE, RecT, Redα and Redβ.

The enzyme mixture may further contain other enzymes, such as helicases and/or nucleic acid repair proteins. In an embodiment, the enzyme mixture comprises an exonuclease, a helicase, a single-stranded DNA binding protein or an annealing protein, and a nucleic acid repair protein.

Homologous Recombination

The homologous recombination technology is well-known in the art. Homologous recombination can be implemented using any methods, any suitable enzymes, enzyme mixtures or enzyme systems known in the art.

An extended linearized vector and a sample containing the target DNA may be brought into contact with an enzyme or an enzyme mixture in a reaction mixture, i.e., incubated in vitro, thereby realizing homologous recombination between the target DNA and the extended linearized vector.

According another aspect of the present application, the vector, the sample containing the target DNA and the abovementioned ligation fragments containing both the target DNA-specific homologous arm and the vector-specific homologous arm can be directly incubated in the reaction mixture together with the enzyme or enzyme mixture, thereby cloning or ligating (e.g., directional cloning or directional ligating) the target DNA into the vector by homologous recombination.

In the aspect of the present application, the vector may be a linearized vector, or may be a circular vector.

The reaction mixture contains a target DNA, a vector and a ligation fragment or an extended linearized vector, an enzyme or an enzyme mixture and a reaction buffer.

In an embodiment, the reaction mixture contains, for example, 1 to 100 mg/L of an exonuclease and 1 to 100 mg/L of a single-stranded DNA binding protein (a single-stranded DNA annealing protein or a strand invasion protein). The reaction mixture may further contain other enzymes, such as a DNA helicase, e.g., 1 to 100 mg/L of the DNA helicase, and/or a nucleic acid repair protein, e.g., 1 to 100 mg/L of the nucleic acid repair protein.

The reaction buffer can be determined according to the enzymes or enzyme mixtures as used, and can be optimized by conventional experimentation.

The reaction buffer comprises buffering agents, salts and ATP, or may comprise Tris, NaCl, EDTA, MgCl₂, glycerol, bovine serum albumin, ATP, phosphate and dithiothreitol. In a preferred embodiment, the reaction buffer comprises: 1 to 10 mg/mL of Tris, 1 to 10 mg/mL of NaCl, 0.1 to 10 mg/mL of EDTA, 0.1 to 10 mg/mL of MgCl₂, 10 to 200 mg/mL of glycerol, 10 to 50 mg/mL of bovine serum albumin (BSA), 0.1 to 10 mg/mL of ATP, 1 to 10 mg/mL of Na₂HPO₄, 0.1 to 10 mg/mL of KH₂PO₄, and 0.1 to 10 mg/mL of dithiothreitol (DTT); the pH value is about 6.8 to about 7.4.

Another aspect of the present application provides a kit for cloning a target DNA into a vector, the kit comprising: (1) an enzyme mixture comprising an exonuclease and a single-stranded binding protein; and (2) a reaction buffer.

The enzyme mixture contains other enzymes. Reference can be made to the part of “enzyme” hereinabove for the exonuclease and the single-stranded binding protein and other enzymes. The reaction buffer can be the reaction buffer mentioned above.

Transformation

The target DNA and a recombination product of the vector can be transformed or transfected into a host cell by a conventional method.

Conventional methods for transformation or transfection include, but are not limited to, calcium phosphate or calcium chloride co-precipitation, electroporation, lipofection, DEAE-dextran-mediated transfection, virus infection, and the like.

The host cell may be a bacterial cell, a fungal cell, a mammalian cell, etc.

Persons skilled in the art can select a suitable method for transformation or transfection and a suitable host cell according to the requirements.

After the transformed host cell is cultured, recombinants containing the target DNA can be screened according to whether a selection marker contained in the vector is present, or can be screened according to the selection marker, e.g., disappearance of resistance (for example, the cloning of the target DNA destroys the resistance gene) under the condition of homologous recombination using a circular vector.

It can be confirmed that the screened cell clones or colonies contain the correct target DNA by, for example, carrying out the PCR reaction on the target DNA.

The operations such as isolation and purification of DNA, selection and transformation of host cells, colony PCR reaction, PCR amplification reaction, and the like are implemented according to the methods described in the standard technology in the art, e.g., Joseph Sambrook et al., Molecular Clonning: A Laboratory Manual, 3^{rd} ed. Cold Spring Harbor Laboratory Press, 2001, and Carl W. Dieffenbach and Gabriela S. Dveksler, PCR primer: A Laboratory Manual, Cold Spring Harbor Laboratory Press, 1995, etc.

The cloning method of the present application can be used for subcloning in which the target DNA is directly cloned, trapping large fragment genes (10 to 100 kb, for example, 10 to 60 kb) in the genomic studies, constructing recombinant plasmids, modifying the bacterial chromosomes, correcting the genes, rapidly constructing general and conditional gene knockout animals (e.g., murines) and studying single nucleotide polymorphism (SNP) (in place of the gene chip technology).

The method of the present application has the following advantages:

1. The method of the present application does not rely on the conventional restriction endonucleases and the unique cleavage sites;
2. The method of the present application is not limited by the molecular size of the target DNA to be cloned;
3. The ligation reaction is carried out in vitro to avoid the contradictions of a low amount and low abundance of the target DNA which is not subjected to the PCR amplification in the entire genome and a low conversion rate caused by co-transfection under the in vivo reaction conditions;
4. The method of the present application has high reaction accuracy;
5. The method of the present application is operated in a simple and convenient, fast and efficient manner.

How to implement the method of the present application is set forth in more details hereinafter through specific examples. However, the method of the present application is not limited to these examples.

### Example 1

The human DHRS4 gene cluster has three copies of genes, which are DHRS4 (15.569 kb), DHRS4L2 (about 35 kb) and DHRS4L1 (also called DHRS4X), respectively, wherein the former two have very high (90% to 98%) homology, and pertain to segmental duplication. The homology between DHRS4L1 and DHRS4 and between DHRS4L1 and DHRS4L2 are 77.8% and 77.7%, respectively. High homology between the three genes limits the application of conventional molecular biology methods, and produces difficulty for the sequencing of the DHRS4 gene (15.569 kb in full length), i.e., it is difficult to carry out accurate sequencing of the gene and the SNP studies through the new-generation gene sequencing technology and the gene chip trapping technology (from Agilent and Nalgene companies).

This example mainly used an enzyme mixture containing RecE and RecT to homologously recombine the DHRS4 gene into a p15A vector (Biovector (Beijing) Co., Ltd.). Briefly, the 15-50 bp sequences at both sides of the DHRS4 gene served as the sequences of the homologous arms, the 20 bp sequences at both sides of the sites in the vicinity of the multiple cloning sites in the p15A vector served as the general PCR amplification primers, and the combination of the two sequences constituted a “combination primer” (i.e., the combination primer=the sequences of the homologous arms from the DHRS4+ the general PCR amplification primers from the p15A vector). Then, PCR amplification was carried out using the p15A plasmid vector as a template and using the high fidelity enzyme Prime STAR MAX DNA polymerase (Takara company) and the “combination primer”. The resulting PCR product (300 ng) and the whole genomic DNA from human blood in a ratio of 1:20-30 (6000 ng to 9000 ng) and a suitable amount (0.5 to 2.0 U) of the enzyme mixture (RecE and RecT were mixed in an equal proportion) were mixed in an Eppendorf tube, incubated for 30 to 60 minutes at 30 to 37° C. and then transformed into a competent Escherichia coli cell JM109. After recovery for 70 minutes at the condition of 37° C., the transformed Escherichia coli was coated onto the LB plate containing 100 μg/ml of ampicillin and cultured overnight at 37° C.

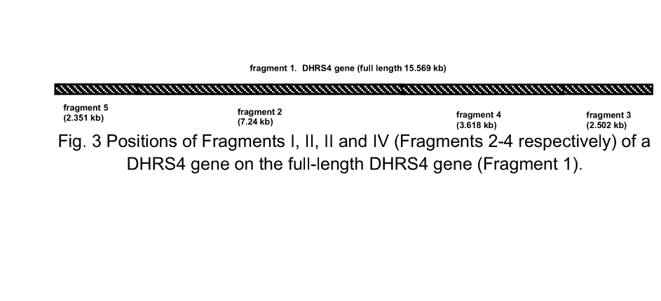

By PCR, the DHRS4 gene on the plasmid extracted from the ampicillin (Amp) resistance monoclonal strain was used as the template for detection by dividing it into four specific fragments or in a full-length fragment (see FIG. 3), the resulting fragments being:

Fragment 1: a DHRS4 full-length gene (15.569 kb); Fragment 2: the DHRS4 gene fragment I (7.24 kb in length); Fragment 3: the DHRS4 gene fragment II (2.502 kb in length); Fragment 4: the DHRS4 gene fragment III (3.618 kb in length); Fragment 5: the DHRS4 gene fragment IV (2.351 kb in length); M: a DNA molecular weight marker.

The primers of the PCR detection reaction were as follows:

Fragment 1: primers of the DHRS4 gene (a full-length gene, 15.569 kb):

upstream primer:

(SEQ ID NO: 1)

5′-TCACCGCCCCTGGGAAGAGTGGAAC-3′
downstream primer:

(SEQ ID NO: 2)

5′-AAGCACCCAACACTGAGAAATGAAC-3′

Fragment 2: primers of the DHRS4 gene fragment I (7.24 kb):

upstream primer:

(SEQ ID NO: 3)

5′-GACAGTAGTATGGTAGACAGAATAG-3′
downstream primer:

(SEQ ID NO: 4)

5′-AGATGCCATGTAGGGCTTTAATAGC-3′

Fragment 3: primers of the DHRS4 gene fragment II (2.502 kb):

upstream primer:

(SEQ ID NO: 5)

5′-CATGAGGATGGGCAGTTTCTTCCCT-3′
downstream primer:

(SEQ ID NO: 6)

5′-AAGCACCCAACACTGAGAAATGAAC-3′

Fragment 4: primers of the DHRS4 gene fragment III (3.618 kb):

upstream primer:

(SEQ ID NO: 7)

5′-GCTATTAAAGCCCTACATGGCATCT-3′
downstream primer:

(SEQ ID NO: 8)

5′-TTACAGGCATGAGCCACCCCACCCA-3′

Fragment 5: primers of the DHRS4 gene fragment IV (2.351 kb):

upstream primer:

(SEQ ID NO: 9)

5′-TCACCGCCCCTGGGAAGAGTGGAAC-3′
downstream primer:

(SEQ ID NO: 10)

5′-CTATTCTGTCTACCATACTACTGTC-3′

The sequences of the “combination primers” were as follows:

upstream primer:

(SEQ ID NO: 11)

5′-
GCGCGGCTTTGAATCCAATTGACCTGTTCATTTCTCAGTGTTGGGTGCTT
tataccgtctagagttaacc-3′
downstream primer 5-3:

(SEQ ID NO: 12)

5′-
GCATGGATCAGACCAGCAAGTATGGGTTCCACTCTTCCCAGGGGCGGTG
Acgtccgcgcggctcgagctt-3′

The primers of Fragment 3 overlapped the primers of Fragment 4 in terms of design, i.e., the PCR product of Fragment 3 overlapped the PCR product of Fragment 4.

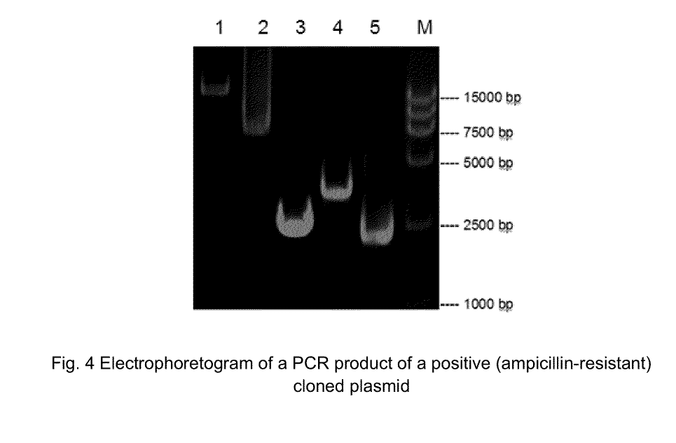

The results of the PCT detection were shown in FIG. 4. The results in FIG. 4 show that over 15 kb of the large DNA fragment was successfully cloned into the vector by the method of the present application.

### Example 2

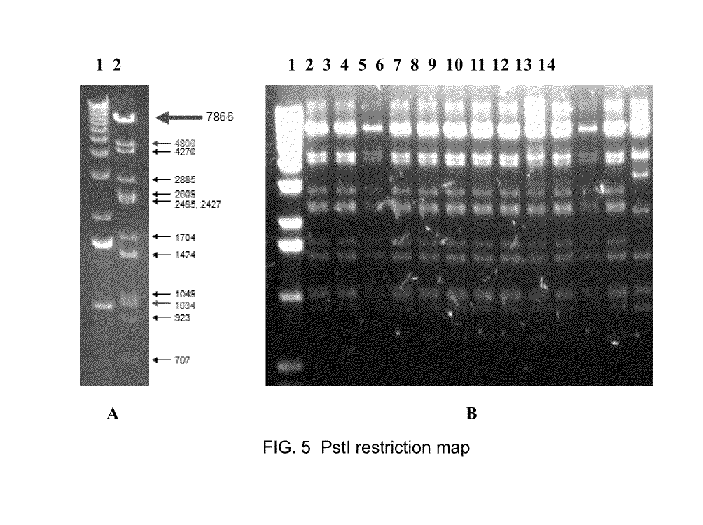

According to the method described in Example 1, suitable PCR primers were designed to clone a murine TFIIA gene (transcription factor IIA) with a length of about 30 kb from plasmid LAWRIST7-mTFIIA (Gene Bridges GmbH) to the p15A vector. The PCR product of the plasmid resulted from positive clones was verified by the PstI restriction map. FIG. 4 provides a restriction map of the PCR product resulted from partially positive clones therein. As can be seen from the FIG. 5, Lanes 2 to 13 in FIG. B were normal ligations, while Lane 14 was non-proper ligation. Statistics show that the rate of successful ligation (the number of the clones that were correctly ligated/the number of the detected positive clones) in accordance with the cloning method of the present application was 65% to 70% or so.

### Example 3

The method of Example 1 was used to clone the target DNA molecules with differing sizes into the vectors. Each group of the rates of correct cloning in Table 1 below were the rates of correct cloning (i.e., rates of successful ligation) verified by restriction detection (a restriction map of the PstI enzyme).

TABLE 1

| Sizes of the | | | | | |
|---|---|---|---|---|---|
| target DNA | 1 kb | 3 kb | 5 kb | 10 kb | 30 kb |
| Numbers of clones | 436 | 615 | 340 | 279 | 185 |
| (amounts of coated | | | | | |
| bacteria in 100 μL) | | | | | |
| Rates of correct | 9/10 | 8/10 | 8/10 | 6/10 | 7/10 |
| cloning | | | | | |

I claim:

1. A method of cloning a target DNA into a vector, the method comprising:
(a) adding to both ends of a linearized vector a first sequence and a second sequence, respectively, wherein the first sequence has a sequence that is homologous with a sequence of a first end of the target DNA or a flank sequence thereof, the second sequence has a sequence that is homologous with a sequence of a second end of the target DNA or a flank sequence thereof, an extended linearized vector with both ends respectively having the first sequence and the second sequence is obtained, and each of the first sequence and the second sequence, independently, has a length of at least 12 nucleotides, preferably 15 to 50 nucleotides, more preferably 35 to 50 nucleotides; and
(b) bringing the extended linearized vector into contact with a sample containing the target DNA, and cloning the target DNA into the vector by homologous recombination.

2. The method according to claim 1, wherein step (a) is carried out as follows:
(i) providing a first primer, a second primer and a vector, wherein the first primer comprises the first sequence as the 5′-end and a sequence as the 3′-end that is specific to a first region of the vector, the second primer comprises the second sequence as the 5′-end and a sequence as the 3′-end that is specific to a second region of the vector, the sequence that is specific to the first region of the vector is preferably a sequence complementary to the first region of the vector, and the sequence that is specific to the second region of the vector is preferably a sequence complementary to the second region of the vector; and
(ii) bringing the first primer and the second primer into contact with the vector as a template, and obtaining the extended linearized vector with both ends respectively having the first sequence and the second sequence by a polymerase chain reaction.

3. The method according to claim 2, wherein the vector as the template in the polymerase chain reaction is a linearized vector, and the first and second regions are preferably a first end and a second end of the linearized vector, respectively.

4. The method according to claim 2, wherein the vector as the template in the polymerase chain reaction is a circular vector.

5. The method according to claim 1, wherein step (a) is carried out as follows:
(i) providing a first ligation fragment, a second ligation fragment and a vector, wherein the first ligation fragment has a sequence homologous with the sequence of the first end of the target DNA or the flank sequence thereof and a sequence homologous with the first region of the vector, and the second ligation fragment has a sequence homologous with the sequence of the second end of the target DNA or the flank sequence thereof and a sequence homologous with the second region of the vector; and
(ii) bringing the first ligation fragment and the second ligation fragment into contact with the vector, and obtaining the extended linearized vector with both ends respectively having the first sequence and the second sequence by homologous recombination.

6. A method of cloning a target DNA into a vector, the method comprising:
(a) providing a first ligation fragment, a second ligation fragment and a vector, wherein the first ligation fragment has a sequence homologous with a first region of the vector and a sequence homologous with a sequence of a first end of the target DNA or a flank sequence thereof, and the second ligation fragment has a sequence homologous with a second region of the vector and a sequence homologous with a flank sequence of a second end of the target DNA; and
(b) bringing a first DNA fragment and a second DNA fragment into contact with a linearized vector and a sample containing the target DNA, and cloning the target DNA into the vector by homologous recombination.

7. The method according to claim 1, wherein the homologous recombination is carried out in the presence of an exonuclease and a single-stranded DNA binding protein or an annealing protein, the exonuclease is preferably Escherichia coli exonuclease I, Escherichia coli exonuclease III, Escherichia coli exonuclease VII, lambda bacteriophage exonuclease, T7 bacteriophage exonuclease, Redα, RecE, or a mixture thereof, and the single-stranded DNA binding protein or the annealing protein is preferably extreme thermostable single-stranded DNA binding protein (ET SSB), RecA, T4 Gene 32 Protein, Thermus thermophilus RecA (Tth RecA), Escherichia coli single-stranded DNA binding protein (SSB), Redβ, RecT, or a mixture thereof.

8. The method according to claim 1, wherein the homologous recombination is carried out in the presence of RecE and RecT or in the presence of Redα and Redβ.

9. The method according to claim 1, wherein the homologous recombination is carried out in the presence of RecE, RecT, Redα and Redβ.

10. A kit for cloning a target DNA into a vector, the kit comprising:
(a) an enzyme mixture comprising an exonuclease and a single-stranded DNA binding protein or an annealing protein; and
(b) a reaction buffer.

11. The kit according to claim 10, wherein the exonuclease is a prokaryote exonuclease or a virus exonuclease, and is preferably Escherichia coli exonuclease I, Escherichia coli exonuclease III, Escherichia coli exonuclease VII, lambda bacteriophage exonuclease, T7 bacteriophage exonuclease, Redα, RecE, or a mixture thereof.

12. The kit according to claim 10, wherein the single-stranded DNA binding protein or the annealing protein is extreme thermostable single-stranded DNA binding protein (ET SSB), RecA, T4 Gene 32 Protein, Thermus thermophilus RecA (Tth RecA), Escherichia coli single-stranded DNA binding protein (SSB), Redβ, RecT, or a mixture thereof.

13. The kit according to claim 10, wherein the enzyme mixture comprises RecE and RecT, or Redα and Redβ.

14. The kit according to claim 10, wherein the enzyme mixture comprises RecE, RecT, Redα and Redβ.

15. The kit according to claim 10, wherein the enzyme mixture further comprises a helicase and/or a nucleic acid repair protein.

16. The kit according to claim 15, wherein the enzyme mixture comprises an exonuclease, a helicase, a single-stranded DNA binding protein or an annealing protein, and a nucleic acid repair protein.

17. The kit according to claim 10, wherein the reaction buffer comprises 1 to 10 mg/mL of Tris, 1 to 10 mg/mL of NaCl, 0.1 to 10 mg/mL of EDTA, 0.1 to 10 mg/mL of MgCl₂, 10 to 200 mg/mL of glycerol, 10 to 50 mg/mL of bovine serum albumin (BSA), 0.1 to 10 mg/mL of ATP, 1 to 10 mg/mL of Na₂HPO₄, 0.1 to 10 mg/mL of KH₂PO₄, and 0.1 to 10 mg/mL of dithiothreitol (DDT); and the pH value is about 6.8 to about 7.4.


SEQUENCE LISTING (12 sequences)

SEQ ID NO: 1 - DNA, 25 bp, Artificial Sequence
  Example 1Fragment 1 (upstream primer)
tcaccgcccctgggaagagtggaac

SEQ ID NO: 2 - DNA, 25 bp, Artificial Sequence
  Example 1Fragment 1 downstream primer
aagcacccaacactgagaaatgaac

SEQ ID NO: 3 - DNA, 25 bp, Artificial Sequence
  example 1 fragment 2 (upstream primer)
gacagtagtatggtagacagaatag

SEQ ID NO: 4 - DNA, 25 bp, Artificial Sequence
  Eample 1 fragment 2 downstream primer
agatgccatgtagggctttaatagc

SEQ ID NO: 5 - DNA, 25 bp, Artificial Sequence
  Example 1 fragment 3 upstream primer
catgaggatgggcagtttcttccct

SEQ ID NO: 6 - DNA, 25 bp, Artificial Sequence
  example 1 fragment 3 downstream primer
aagcacccaacactgagaaatgaac

SEQ ID NO: 7 - DNA, 25 bp, Artificial Sequence
  Example 1 fragment 4 upstream primer
gctattaaagccctacatggcatct

SEQ ID NO: 8 - DNA, 25 bp, Artificial Sequence
  Example 1 fragment 4 downstream primer
ttacaggcatgagccaccccaccca

SEQ ID NO: 9 - DNA, 25 bp, Artificial Sequence
  Example 1 fragment 5 upstream primer
tcaccgcccctgggaagagtggaac

SEQ ID NO: 10 - DNA, 25 bp, Artificial Sequence
  Example 1 Fragment 4 downstream primer
ctattctgtctaccatactactgtc

SEQ ID NO: 11 - DNA, 70 bp, Artificial Sequence
  Example 1 combination primers upstream primer
gcgcggctttgaatccaattgacctgttcatttctcagtgttgggtgctttataccgtctagagttaacc

SEQ ID NO: 12 - DNA, 70 bp, Artificial Sequence
  Example 1 combination primers downstream primer
gcatggatcagaccagcaagtatgggttccactcttcccaggggcggtgacgtccgcgcggctcgagctt